(19) United States
(12) Patent Application Publication
(10) Pub. No.: US 2015/0313675 A1
(43) Pub. Date: Nov. 5, 2015

(54) System and Device for A Fully Automated Approach to Facilitate Stellate Ganglion Block or Pulsed Radiofrequency Procedure for Treating PTSD

(71) Applicant: Eugene Lipov, Hoffman Estates, IL (US)

(72) Inventor: Eugene Lipov, Hoffman Estates, IL (US)

(21) Appl. No.: 14/702,281

(22) Filed: May 1, 2015

Related U.S. Application Data
(60) Provisional application No. 61/987,231, filed on May 1, 2014.

(51) Int. Cl. A61B 19/00; A61B 8/08; A61B 5/00; A61N 1/40; A61M 5/19; A61M 5/32; A61M 19/00; A61M 5/315; A61B 6/12; A61M 5/31

(52) U.S. Cl. CPC A61B 19/22; A61B 6/12; A61B 8/0841; A61B 5/0059; A61M 5/31; A61M 5/19; A61M 5/329; A61M 19/00; A61M 5/3158; A61N 1/40; A61M 2210/1003

(57) ABSTRACT

A system and method to facilitate a stellate ganglion block or a pulsed radiofrequency procedure to treat post-traumatic stress disorder. The method can be fully automated and guided by a computer or the process can be guided by the surgeon. A computer including a central processing unit (CPU) is connected to a body scanning device and an injection mechanism. The body scanning device is used to read images of a patient's body. The information collected from the images is transmitted to the CPU. A housing for the injection mechanism encloses syringes for delivery of an anesthetic and a clonidine mixture. After the anesthetic is injected into the patient's skin from a first syringe, the housing is relocated by a connecting arm and a second syringe moves a needle into contact with the patient's bone. An electric eye scans for blood before the clonidine mixture is injected by the second syringe.

This application claims priority to provisional application Ser. No. 61/987,231 filed May 1, 2014, to the extent allowed by law.

### BACKGROUND OF THE INVENTION

1. Field of the Invention

The present invention is directed to a system and method for facilitating a stellate ganglion block or C-6, C-7, or C-3 procedure to be performed in the treatment of Post-Traumatic Stress Disorder (PTSD) easily and safely. More particularly the invention is directed to a process to perform stellate ganglion block. that is safer and saves the training time of a practitioner to practice the procedure.

2. Description of the Prior Art

Stellate ganglion is formed by the fusion of the neck and upper thoracic ganglion. Stellate ganglion block (SGB) anesthesia is widely used for treating patients with head, face, chest and back pain. Currently, the clinical use of SGB is a commonly used anesthetic technique that has traditionally been administered for pain relief. The general anesthetic injection needle requires the surgeon to perform the carotid artery sheath separation with his/her left hand, and guide the needle with his/her right hand holding the syringe to position the needle vertically and gently pushing the syringe, until the needle arrives at the cervical bone. Then the surgeon will pull the tip of the needle back 2 mm, and withdraw the piston to confirm that there is no blood or cerebrospinal fluid. The surgeon then proceeds to inject the anesthetic. During the process of pulling the tip of the needle back and withdrawing the piston, the position of the syringe is subject to undesirable change and shift, due to the lack of support and position tagging or labeling.

The shift and repositioning of the needle is potentially harmful to the surrounding blood vessels, nerves and lymphatic vessels, and can even lead to incorrect anesthetic injection, where the result could be serious complications, endangering the lives of patients. The operating surgeon is also highly trained in invasive procedures. Extensive training is thus required for the surgeons performing the invasive procedures to ensure safety.

SGB (for the purpose of this document, refers to C6 and C7 as well as C3 instead of commonly recognized as only C7) is also recently recognized as an effective treatment for PTSD. PTSD is a devastating and complex pathological anxiety condition. Patients often suffer from severe distress and impairment in mental and physical functioning. Symptoms of PTSD typically include intense anxiety, hyper-arousal, flashbacks and sleep disturbances. PTSD is also considered a public health dilemma because nearly 80% of the population experience traumatic events in their lifetime.

For individuals impacted with PTSD, medical intervention is usually recommended or required. Currently, standard treatments for PTSD include pharmacotherapy and psychotherapy. Tests in over 500 patients using SGB to resolve PTSD demonstrate the effect in thirty minutes. Treatment of PTSD is considered successful if there is a reduction in the severity, frequency and intensity of symptoms. Approximately 10% of PTSD affected patients seek treatment within twelve months of the PTSD stimulating traumatic event. The occurrence of PTSD in the US military has been repeatedly emphasized, where the U.S. military suicide rate is over 22 per day, compared to the prior rate cited by the Department of Veterans Affairs at 18 per day. Possibly up to 30% of veterans returning from combat areas suffer from PTSD and over 400,000 veterans receive disability benefits due to PTSD. The need to treat PTSD is immediate and immense. Over 1,000,000 military and over 25,000,000 non-military people need to be treated in the U.S. alone. The Department of Veterans Affairs and other military institutions do not have the specialized personnel to treat this large number of patients with SGB due to the extensive training required by the procedures.

### SUMMARY OF THE INVENTION

The present invention comprises a system that includes a body scanning device and an injection mechanism and a process that can be fully automated and guided by a computer or a process that can be guided by a surgeon. The system comprises a central processing unit (CPU) connected to the body scanning device, such as an ultrasound device or an x-ray device, with or without a laser, and the injection mechanism. The body scanning device is used to read the image of a patient's body. The information collected by the reading from the body scanning device is transmitted to the CPU, with or without a laser. A housing for the injection mechanism encloses syringes for delivery of an anesthetic and a clonidine mixture to a patient in need of treatment. The syringe bodies are driven by gears during injection and refraction. The syringe plungers are also gear driven to inject the medication from the syringe bodies and into the corresponding needles. After the anesthetic is injected into the patients skin from one syringe, the housing is relocated by a mechanical arm, and a second syringe body moves a needle into contact with the patient's bone. An electric eye scans for blood before the clonidine mixture is injected by the second syringe body. The entire SGB process is automated and guided by the CPU, with or without the assistance of a scanning laser.

### BRIEF DESCRIPTION OF THE DRAWINGS

The features of the present invention which are believed to be novel are set forth with particularity in the appended claims. The invention may best be understood from the following detailed description of currently illustrated embodiments thereof taken in conjunction with the accompanying drawings wherein like numerals refer to like parts, and in which:

FIG. 1 is a schematic illustration of a first embodiment, showing a general layout of the automated SGB system of the present invention adjacent a patient to be treated.

FIG. 2 is an illustration of the first embodiment, showing placement of the ultrasound device and injection mechanism adjacent the patient's body.

FIG. 3 is a schematic illustration of the first embodiment, showing injection operation by the use of the injection mechanism with detailed components inside the injection housing.

FIG. 4 illustrates a second embodiment of the present invention using pulsed radiofrequency inside the injection housing.

FIG. 4A provides a detailed illustration of the second embodiment, showing the needle of the pulsed radiofrequency embodiment.

FIG. 5 is a schematic illustration of a third embodiment, showing a general layout of the automated SGB system of the present invention adjacent to a patient to be treated.

FIG. 6 is a flow diagram of a method of the third embodiment of the present invention.

FIG. 7A is a schematic illustration of the third embodiment of the present invention, showing the body scanning device positioned above the patient's neck.

FIG. 7B is a detail view of a schematic illustration of the third embodiment of the present invention, showing the body scanning device positioned on the patient's neck.

FIG. 8 is a schematic illustration of the third embodiment of the present invention, showing the injection mechanism positioned above the patient's neck.

FIG. 9A is a detail view of the injection mechanism of the third embodiment of the present invention.

FIG. 9B is a detail view of the injection mechanism of the third embodiment of the present invention, showing force sensors.

FIG. 10A is a detail view of the injection mechanism of the third embodiment of the present invention, showing the advancement of the needle to the patient's bone.

FIG. 10B is a detail view of the injection mechanism of the third embodiment of the present invention, showing the needle pull back position to look for blood.

FIG. 10C is a detail view of the injection mechanism of the third embodiment of the present invention, showing the injection of bupivacaine/clonidine into the patient's bone.

FIG. 11 is a detail view of the injection mechanism of the third embodiment of the present invention, showing all needles removed from the patient's neck.

FIG. 12 is a perspective view of the monitor display screen of the third embodiment of the present invention.

FIG. 13 is a detail view of the injection mechanism of the third embodiment of the present invention.

FIG. 14 is a detail view of a non-rigid, snake-like arm of a fourth embodiment of the present invention.

### DETAILED DESCRIPTION OF THE ILLUSTRATED EMBODIMENTS

Referring to FIG. 1, there is shown an illustrated embodiment of the automated SGB system according to the present invention. A computer 100 has a CPU which stores and processes relevant images and information collected and used in the entire SGB process. The computer 100 is electronically connected to a body scanning device 102 and injection mechanism 104. The body scanning device 102 in the illustrated embodiment is an ultrasound scanner. In another embodiment of the present invention, the body scanning device 102 is an X-ray scanner. The ultrasound device uses high frequency sound waves to create an image of the fusion of the neck and upper thoracic ganglion of a patient 106. The body scanning device 102 collects images and data to assist surgeons in performing the SGB procedures at C6 or C7, with or without an injection at the C3 level. The collected information is transmitted from the body scanning device 102 to the CPU of the computer 100 for processing.

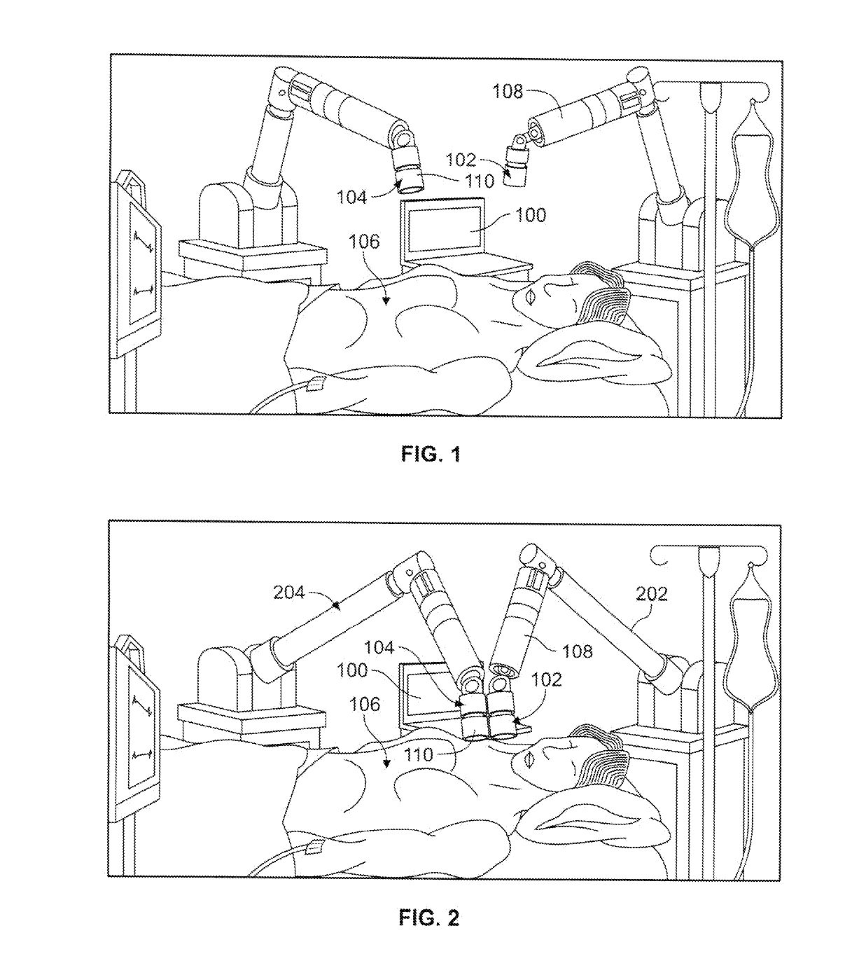

The stellate injection process starts as illustrated in FIG. 2. The process can be fully automated and guided by the computer 500 or the process can be guided by the surgeon. A mechanical arm 108 places the body scanning device 102 around the patient's 106 middle cervical ganglion area and/or stellate ganglion area. The surgeon uses the guidance provided by the body scanning device 102 to identify the structures in the neck area for placement of the injection. In the illustrated embodiment in FIG. 2, the housing 110 for the injection mechanism 104 is also located at the patient's 106 neck area adjacent to the body scanning device 102. The housing 110 for the injection mechanism 104 is placed at the designated position by an adjustable connecting arm 204. Both the body scanning device 102 and the injection mechanism 104 are electronically connected to the CPU 100 that collects information and provides guidance for the injection process.

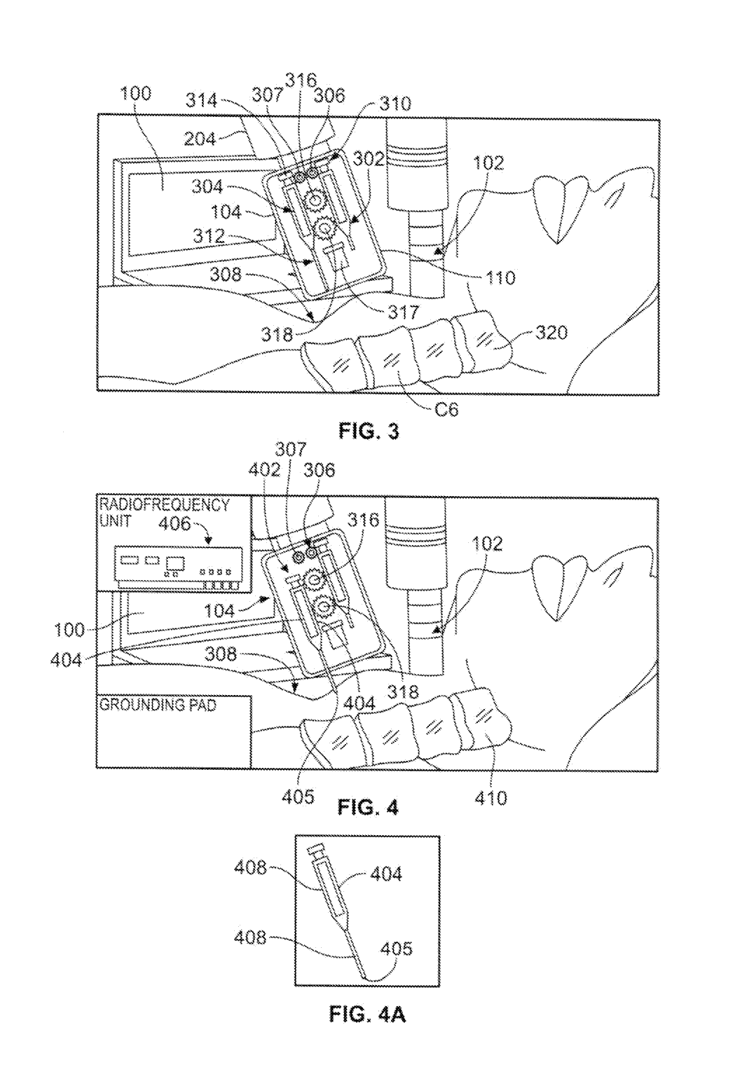

The injection mechanism 104 is further detailed in FIG. 3. The body scanning device 102 and the injection mechanism housing 110 are both in contact with the surface of the patient's skin 308 at a location defined by the body scan image and information in the CPU 100. The injection mechanism housing 110 encloses two syringes, first syringe 302 and second syringe 304. The first syringe 302 has a smaller needle than the second syringe 304, which can be smaller in gauge and/or in length. The first syringe 302 contains short acting anesthetics for subcutaneous injection, to produce local anesthesia. An example of the anesthetics contained in first syringe 302 is lidocaine. The first syringe 302, in the illustrated embodiment, has a short small needle, approximately 5/9^{th} of an inch, from twenty-five to thirty gauge. Syringes 302, 304 are movable relative to housing 110, as will be explained.

The second syringe 304 has a twenty-two to twenty-five gauge needle and is filled with long acting anesthetic or the mixture of anesthetic and clonidine. Gears 306 and 307 are used to power the movement of the plungers in each syringe, first syringe 302 and second syringe 304 respectively. Both syringes 302 and 304 are attached to respective needles that will penetrate through the patent's skin for the injections. Gears 316 and 318 are operationally connected to respective syringes 302, 304 to move the syringes axially with respect to housing 110. When gears 316, 318 move the positions of syringes 302, 304, their attached needles are deployed or retracted out of or into the housing 110 with the movements of syringes 302, 304. When the first syringe 302 is advanced by gear 316, the tip of the needle penetrates through the surface of the patient's skin 308. The first plunger 310 of the first syringe 302 is then forced down by gear 306 to inject about three cubic centimeters (cc) of lidocaine under the patient's skin 308, in an illustrated embodiment of the procedure. Gear 316 then retracts the first syringe 302 to remove the tip of the needle from the patient's skin. The injection of local anesthesia is then complete, and signals of the anesthesia injection process are transmitted to the CPU at computer 100 at the completion of the anesthesia injection.

The injections may be administered at cervical bone C6, cervical bone C7, cervical bone C3, or any combination thereof. The injection may be administered higher than C-6, for instance at C-3, on the right or left side. The injection may also be administered lower than C-6, for instance at C-7, on the right or left side. The injections may be administered at multiple cervical targets in the same instance of administration. The administration of the injections may also be at C-6 and/or C-7 followed by administration of an injection at C-3. A C-3 injection turns off sympathetic nerves, the fight or flight nerves, in a part of the brain different than the stellate ganglion block. Combining a C-6 injection followed by a C-3 injection, a C-7 injection followed by a C-3 injection, or a C-6 and C-7 injection followed by a C-3 injection turns off almost all of the sympathetic nerves.

The mechanical arm 204 adjustably supporting the injection housing 110 then glides over the patient's skin 308 to reposition the second syringe 304 to a position where the needle 312 is pointing at cervical bone C6, C7, or C3. The repositioning is guided by the computer 100 with the assistance of the body scanning device 102, such as an ultrasound scanner or X-ray scanner. Gear 318 advances the second syringe 304 down to where the needle 312 penetrates the patient's skin 308. The tip of the needle 312 is advanced and guided until the needle 312 arrives at the bone area 320, in particular at cervical bone C6, C7, or C3.

The gear 318 will next retract and guide the second syringe 304 to pull the needle tip 312 backwards for about one mm after the needle tip 312 reaches the bone 320. The computer 100 controls the moving of the second plunger 314 and commands the second plunger 314 to withdraw for a short distance, drawing any substance that potentially could be drawn. An electric eye 317 enclosed by the injection housing 110 is connected to the computer 100 to provide images of the fluid in second syringe 304 through the clear walls of the second syringe 304. The CPU can then inspect whether the withdrawal of the second plunger 314 brought any blood into the second syringe 304. During the conventional SGB process, the surgeon is also required to check if any cerebrospinal fluid is drawn into the syringe during the withdrawal process. However, with the images produced by the body scanning device 102, the needle tip 312 is positioned more accurately, and therefore the chances of drawing cerebrospinal fluid and blood into second syringe 304 are significantly reduced or eliminated.

When the surgeon confirms that there is no blood drawn into second syringe 304, the surgeon will issue the command through computer 100 to advance the second plunger 314 to inject the fluid contained in second syringe 304 into the subcutaneous area adjacent bone C6, C7, or C3. In the illustrated embodiment, the second syringe 304 is filled with long acting anesthetic, such as bupivacaine, or the mixture of anesthetic and clonidine, in an amount around seven cc. The gear 318 then retracts the second syringe 304 into housing 110 to remove the tip of the needle from the patient's body. The entire automated SGB process is then complete.

The modified stellate injection used and described previously in this document is a C-6, or middle cervical ganglion injection. It is safer to perform this described procedure than the conventional SGB procedures for two reasons. First, C6 is higher than C7 (current location of SGB) thus being farther from the lung, and therefore reducing the chance of a pneumothorax. Also the vertebral artery, which injection is a common cause of seizures, is in a bony canal whereas in C7 it is exposed. Stellate ganglion block anesthesia is widely used for treating patients with head, face, chest and back pain. Currently, the clinical use of stellate ganglion block (SGB) is a commonly used anesthetic technique that has traditionally been administered for pain relief.

A second embodiment of the present invention uses pulsed radiofrequency instead of the second injection described previously to treat PTSD. As shown in FIG. 4, a pulsed radiofrequency delivery device 402 is enclosed in housing 110, in place of the second syringe 304 illustrated in FIG. 3. The pulsed radiofrequency delivery device 402 is connected with the radiofrequency generating unit 406, which is further connected with and controlled by the computer 100 and to a grounding pad 410. The pulsed radiofrequency delivery device 402 is attached to a twenty-two gauge, ten cm long insulated needle 404, with a ten mm, 42° C. active needle tip 405. The gear 318 advances the pulsed radiofrequency delivery device 402 down to penetrate the needle 404 through the patient's skin 308, and further guide the needle tip 405 until needle tip 405 arrives at bone area 320, in particular at cervical bone C6, C7, C3, or a combination thereof, placing needle tip 405 in the anterior lateral position. The positioning of needle tip 405 is also confirmed by images generated by the body scanning device 102.

A detailed illustration of the pulsed radiofrequency delivery device 402 is shown in FIG. 4A. The needle tip 405 is un-insulated, whereas the upper sections of the needle 404 are insulated.

The pulsed radiofrequency delivery device 402 delivers a 500 kHz pulse, applied at two bursts per second, with each burst lasting twenty milliseconds, for a total of 360 seconds. Other specifications can also be used on the same device.

In a further embodiment, the local anesthetic applied by syringe 302 may be applied by a patch in a manner that is known in the art. The application of local anesthetic by a patch may be utilized in the embodiment of both FIG. 3 and FIG. 4 described above.

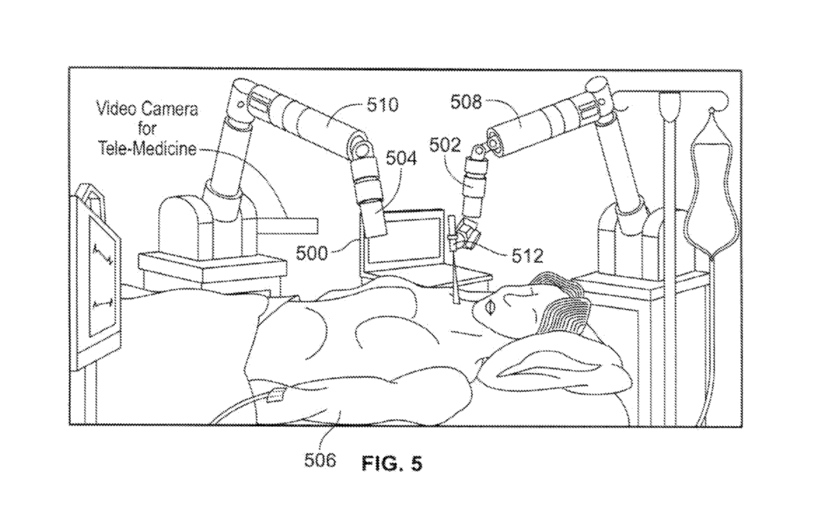

A third embodiment of the present invention, shown in FIGS. 5-13, comprises a computer 500 that includes a CPU which stores and processes relevant images and information collected and used in the SGB process. The computer 500 is electronically connected to a body scanning device, such as an ultrasound probe 502, attached to mechanical arm 508 and an injection mechanism 504 attached to an adjustable connecting arm 510. The injection mechanism includes a disposable box with medications. The ultrasound probe 502 collects images and data to assist surgeons in performing SGB procedures at C6 or C7, with or without an injection at the C3 level. The collected information is transmitted from the ultrasound probe 502 to the CPU of the computer 500 for processing and are displayed on a monitor display screen 522, shown in FIG. 12.

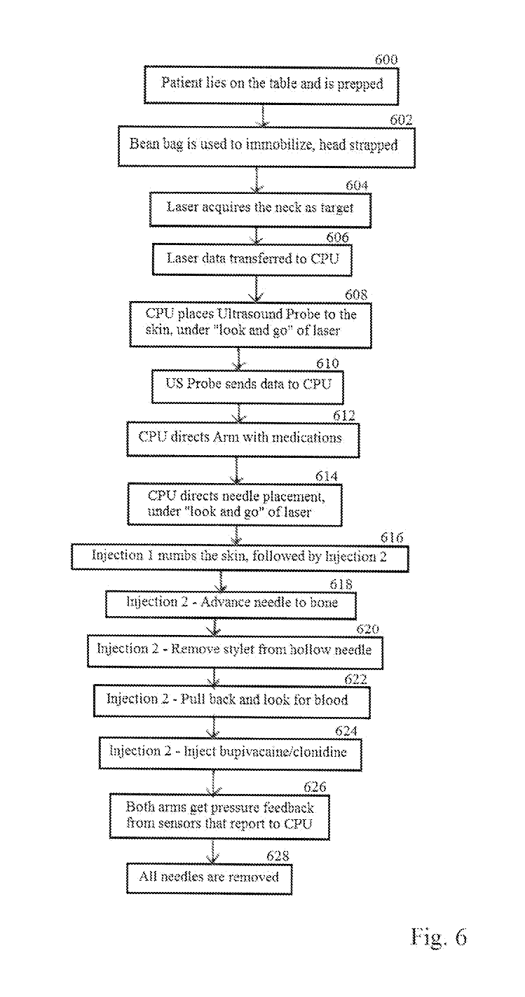

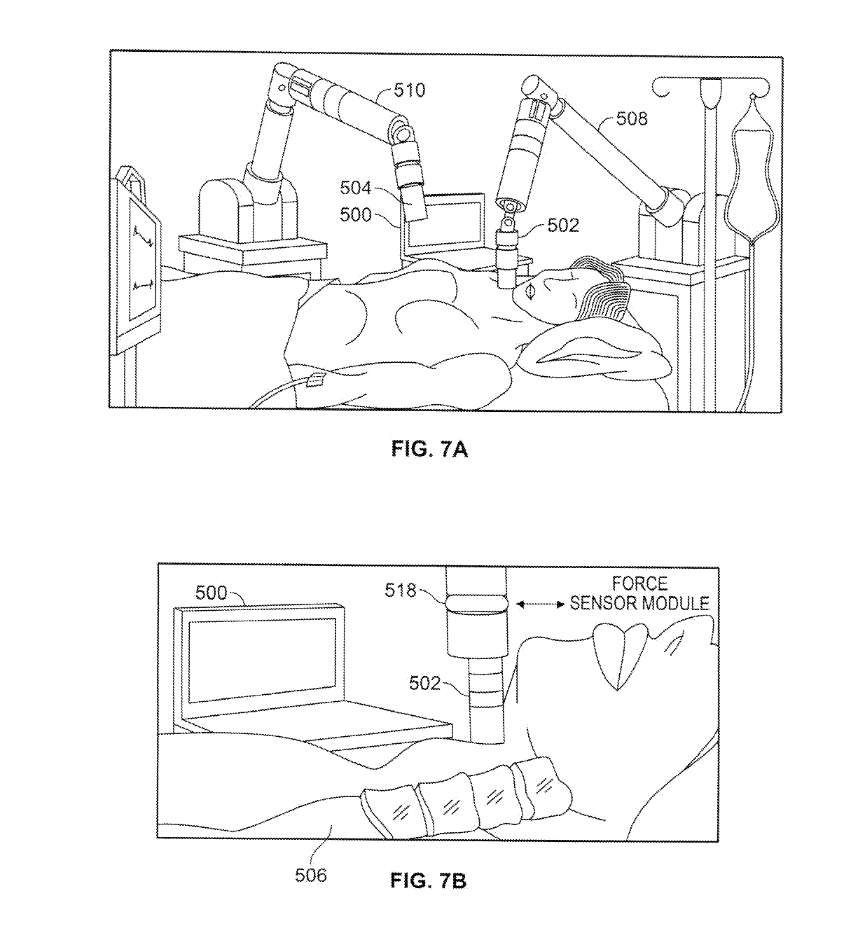

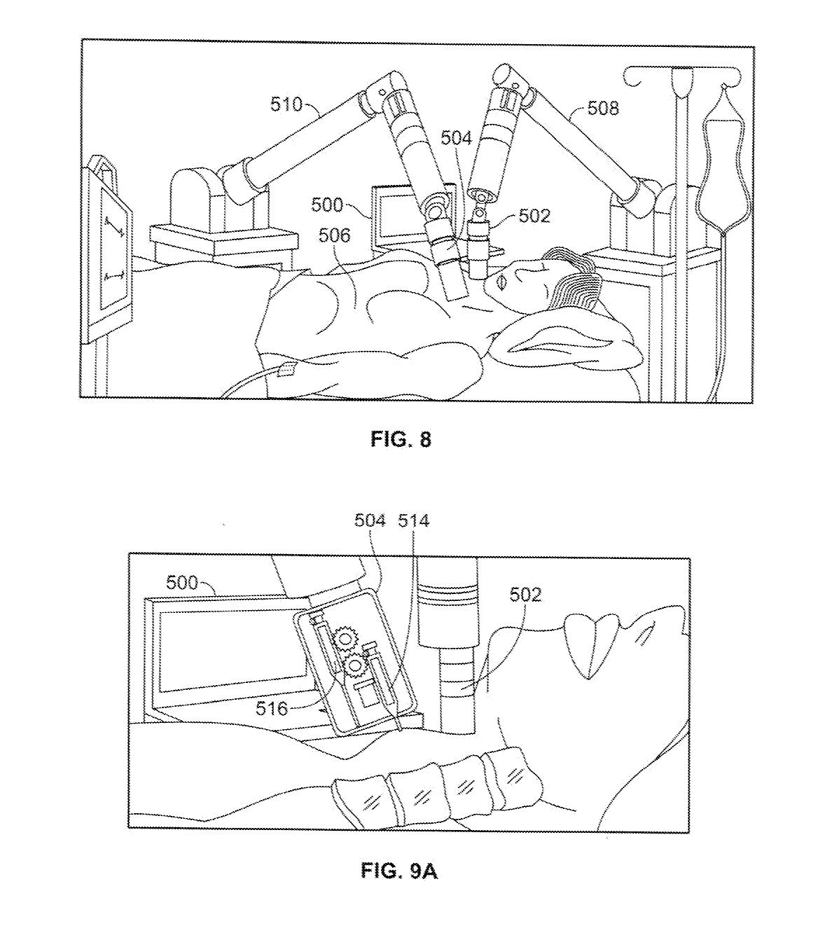

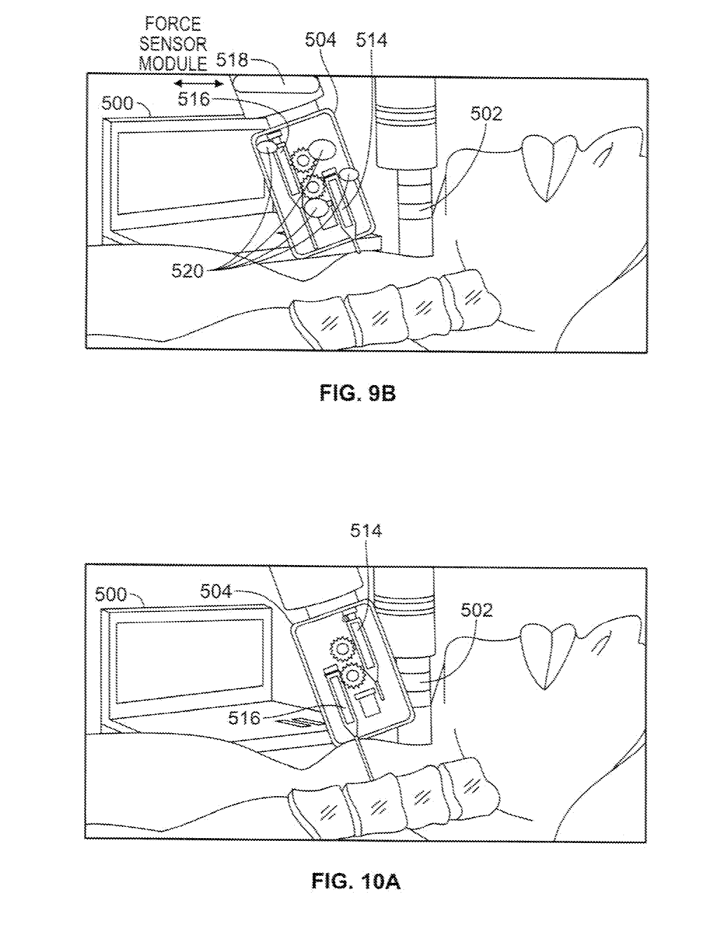

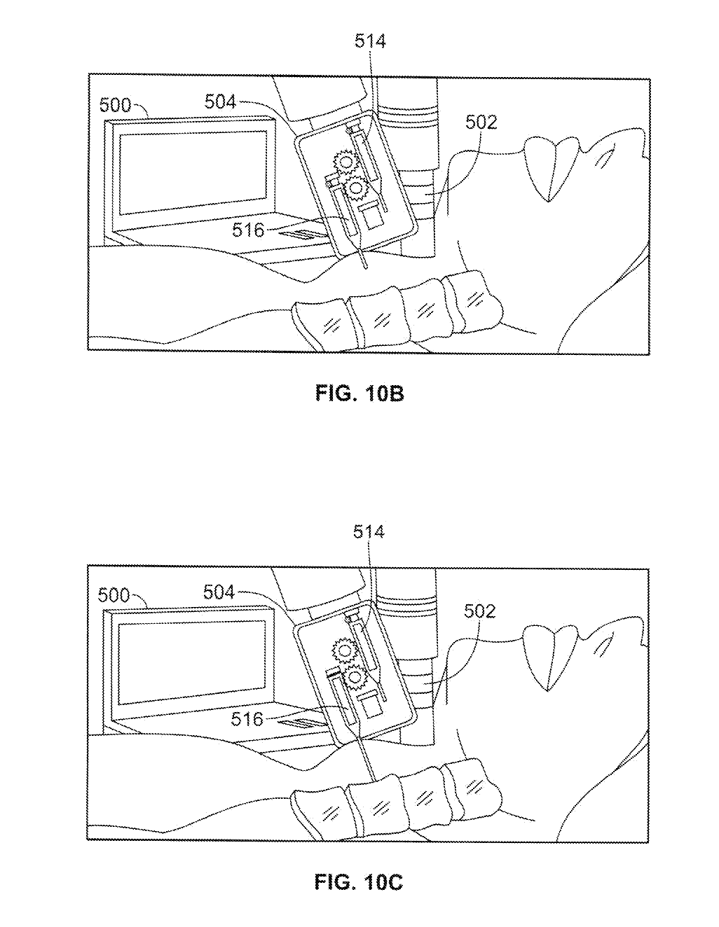

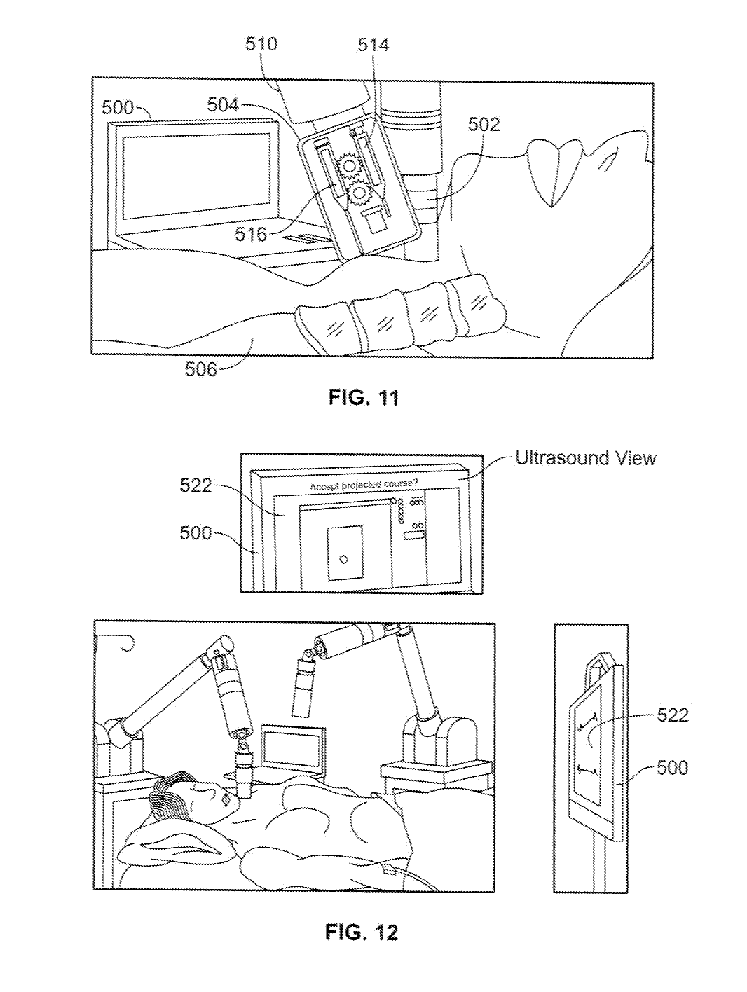

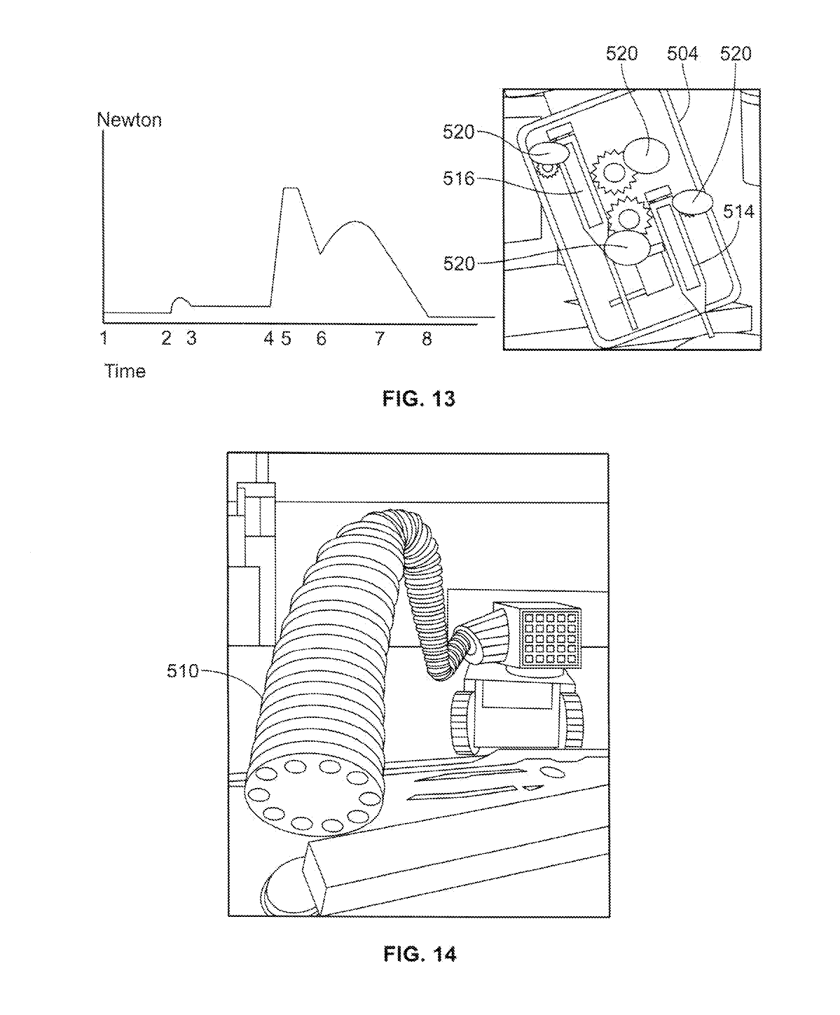

The stellate ganglion process is illustrated in the flow diagram shown in FIG. 6. The process can be fully automated and guided by the computer 500 or the process can be guided by the surgeon. The patient 506 is prepped lying on the table 600. A bean bag is used to immobilize the patient 506 and the patient's head is strapped 602. Laser 512 acquires the neck as the target 604 and the laser data is transferred to the CPU 606. The CPU places the ultrasound probe 502 to the patient's skin 608, shown in FIGS. 7A and 7B, using the “look and go” of laser 512. The ultrasound probe 502 sends the data to the CPU 610. The CPU directs an adjustable connecting arm 510 with the medications 612 and the CPU directs needle placement 614, shown in FIG. 8, using the “look and go” of laser 512. A first injection from a first syringe 514 numbs the skin, shown in FIGS. 9A and 9B, and is following by a second injection 616 from a second syringe 516. The second injection advances the needles to the bone 618 (FIG. 10A), removes the stylet from the hollow needle 620, pulls back the needle and looks for blood 622 (FIG. 10B), and injects bupivacaine or a mixture of bupivacaine and clonidine 624 (FIG. 10C). Both the mechanical arm 508 and the adjustable connecting arm 510 include a force sensor module 518 that obtains pressure feedback from sensors 520 (FIGS. 9B, 13) that report to the CPU 626. All needles are removed 628, shown in FIG. 11.

In a fourth embodiment, shown in FIG. 14, the mechanical arm 108/508 and the adjustable connecting arm 204/510 can comprise a non-rigid, snake-like structure.

The procedure and apparatus described herein may also be used to reduce the incidents of death after polytrauma, improve polytrauma survival which has been demonstrated in the past following SGB, and to treat patients after acute post-traumatic psychiatric injury, which has been demonstrated in a live model.

While one particular embodiment of a system and device for SGB or PRF procedures for treating PTSD of the present invention has been shown and described, it will be apparent to those skilled in the art that changes and modifications may be made without departing from the true spirit and scope of the present invention. It is the intent of the appended claims to cover all such changes and modifications as fall within the true spirit and scope of the invention.

The foregoing description of an illustrated embodiment of the invention has been presented for purposes of illustration and description, and is not intended to be exhaustive or to limit the invention to the precise form disclosed. The description was selected to best explain the principles of the invention and practical application of these principles to enable others skilled in the art to best utilize the invention in various embodiments and various modifications as are suited to the particular use contemplated. It is intended that the scope of the invention not be limited by the specification, but be defined by the claims set forth below.

What is claimed is:

1. A system for facilitating a stellate ganglion block, comprising:
a. a computer including a central processing unit (CPU) adapted to process and store images and information;
b. a body scanning device in communication with the computer and the CPU, said body scanning device adapted to create an image of the fusion of a neck and an upper thoracic ganglion of an individual; and
c. an injection mechanism in communication with the computer and the CPU, said injection mechanism adapted to administer at least one injection to the individual.

2. The system of claim 1, wherein the body scanning device is an ultrasound device.

3. The system of claim 1, wherein the body scanning device is an x-ray device.

4. The system of claim 1, wherein the body scanning device is adapted to collect images and data and transmit the images and data to the CPU of the computer.

5. The system of claim 1, wherein the injection mechanism includes a housing.

6. The system of claim 5, further comprising one of a snake-like device and a mechanical arm adapted to move the body scanning device and place the body scanning device around one of the middle cervical ganglion area and the stellate ganglion area of the individual.

7. The system of claim 6, further comprising one of an adjustable snake-like device and an adjustable connecting arm adapted to support the housing and to move the injection mechanism and place the injection mechanism at a designated position.

8. The system of claim 5, wherein the housing includes a first syringe and a second syringe movable within said housing, said first syringe including a first needle and said second syringe including a second needle.

9. The system of claim 8, wherein at least one of the first length and the first gauge of the first needle is smaller than at least one of the second length and the second gauge of the second needle.

10. The system of claim 8, wherein the first syringe contains a short-acting anesthetic.

11. The system of claim 10, wherein the short-acting anesthetic is lidocaine.

12. The system of claim 8, wherein the first needle is at least 25 gauge.

13. The system of claim 8, wherein the second needle is at least 22 gauge.

14. The system of claim 8, wherein the second syringe contains at least one of a long-acting anesthetic and a mixture of anesthetic and clonidine.

15. The system of claim 5, wherein the housing includes a first gear and a second gear, said first gear adapted to move a first plunger in the first syringe and said second gear adapted to move a second plunger in the second syringe.

16. The system of claim 8, further comprising a third gear and a fourth gear:
a. said third gear in communication with the first syringe and said fourth gear in communication with the second syringe; and
b. said third gear adapted to move the first syringe axially with respect to the housing and deploy and retract the first needle from the housing and said fourth gear adapted to move the second syringe axially with respect to the housing and deploy and retract the second needle from the housing.

17. The system of claim 8, wherein the adjustable connecting arm is adapted to move and position the first syringe and the second syringe over the skin of the individual.

18. The system of claim 8, wherein the housing includes an electric eye in communication with the computer, said electric eye adapted to provide an image of the contents of at least one of the first syringe and the second syringe.

19. The system of claim 5, wherein the housing includes a syringe and pulsed radiofrequency delivery device in communication with a radiofrequency generating unit controlled by the computer.

20. The system of claim 19, wherein the pulsed radiofrequency delivery device includes a needle.

21. The system of claim 20, wherein the housing includes a gear adapted to advance the pulsed radiofrequency delivery device down to penetrate the insulated needle through the skin of the individual.

22. The system of claim 20, wherein the insulated needle is at least 22 gauge.

23. The system of claim 1, further comprising a local anesthetic patch adapted to be applied to the individual.

24. The system of claim 19, wherein the tip of the needle is un-insulated and the upper section of the needle is insulated.

25. A method for facilitating a stellate ganglion block on an individual, said method comprising the steps of:
a. a mechanical arm placing a body scanning device around one of a middle cervical ganglion area and a stellate ganglion area of the individual;
b. identifying the structures in the neck area of the individual for placement of at least one injection;
c. the body scanning device collecting at least one neck image and a plurality of neck data of the individual;
d. transmitting the at least one neck image and plurality of neck data to a central processing unit (CPU) of a computer;
e. the computer processing and storing the at least one neck image and plurality of neck data;
f. an adjustable connecting arm placing a housing of an injection mechanism at a predetermined position on the individual;
g. inserting a first needle of a first syringe at the predetermined position on the individual;
h. injecting a local anesthetic from the first syringe through the first needle at the predetermined position on the individual; and
i. the adjustable connecting arm adjustably supporting the housing repositioning said housing over at least one of a cervical bone C6 of the individual, a cervical bone C7 of the individual, and a cervical bone C3 of the individual.

26. The method of claim 25, further comprising the steps of:
a. inserting a second needle of a second syringe at at least one of the cervical bone C6 of the individual, the cervical bone C7 of the individual, and the cervical bone C3 of the individual;
b. drawing a substance through the second needle into the second syringe within the housing when a substance is present;
c. an electric eye collecting at least one second syringe image showing at least one of the second syringe and the substance, said electric eye in communication with the computer;
d. transmitting the at least one second syringe image to the CPU;
e. the CPU inspecting the at least one second syringe and determining when the second syringe image shows the substance is blood; and
f. injecting one of a long acting anesthetic and a mixture of anesthetic and clonidine from a second syringe into the subcutaneous area adjacent to at least one of the cervical bone C6 of the individual, the cervical bone C7 of the individual, and the cervical bone C3 of the individual when the substance is not blood.

27. The method of claim 25, further comprising the steps of:
a. inserting a second needle of a pulsed radiofrequency delivery device connected to a radiofrequency generating unit at at least one of the cervical bone C6 of the individual, the cervical bone C7 of the individual, and the cervical bone C3 of the individual, placing said second needle in an anterior lateral position;
b. the body scanning device collecting at least one second neck image of the individual;
c. confirming the position of the second needle using the at least one second neck image; and
d. the pulsed radiofrequency delivery device delivering a pulse to the individual.

28. The method of claim 27, wherein the pulse is 500 kHz.

29. The method of claim 27, wherein the pulse is applied at two bursts per second, each burst lasting twenty milliseconds for a total of 360 seconds.

30. The method of claim 26, wherein the long acting anesthetic is bupivacaine.

31. The method of claim 25, further comprising the step of applying a local anesthetic patch to the individual.

32. The system of claim 8, wherein the first needle is 5/9 inch long.

33. The method of claim 27, wherein the pulse is delivered to the sympathetic ganglion of the individual.

34. The method of claim 27, further comprising the step of injecting at least one of bupivacaine and clonidine from the second needle into the individual.

35. The system of claim 1, further comprising a laser scanner adapted to acquire data from the neck of the patient, guide the placement of the body scanning device, and guide the placement of the at least one injection.

36. A method for facilitating a stellate ganglion block on an individual, said method comprising the steps of:
a. a laser scanner obtaining laser data about the neck of the individual;
b. transmitting the laser data to a central processing unit (CPU) of a computer;
c. a mechanical arm placing a body scanning device to the skin of the individual;
d. the body scanning device collecting a plurality of neck data of the individual;
e. transmitting the plurality of neck data to the CPU;
f. an adjustable connecting arm placing a housing of an injection mechanism at a predetermined position on the individual;
g. inserting a first needle of a first syringe at the predetermined position on the individual;
h. injecting a local anesthetic from the first syringe through the first needle at the predetermined position on the individual, said local anesthetic adapted to numb the skin; and
i. the adjustable connecting arm adjustably supporting the housing repositioning said housing over one of a cervical bone C6 of the individual, a cervical bone C7 of the individual, and a cervical bone C3 of the individual.

37. The method of claim 36, further comprising the steps of:
a. inserting a second needle of a second syringe at at least one of the cervical bone C6 of the individual, the cervical bone C7 of the individual, and the cervical bone C3 of the individual;
b. drawing a substance through the second needle into the second syringe within the housing when a substance is present;
c. an electric eye collecting at least one second syringe image showing at least one of the second syringe and the substance, said electric eye in communication with the computer;
d. transmitting the at least one second syringe image to the CPU;
e. the CPU inspecting the at least one second syringe and determining when the second syringe image shows the substance is blood; and
f. injecting one of a long acting anesthetic and a mixture of anesthetic and clonidine from a second syringe into the subcutaneous area adjacent to at least one of the cervical bone C6 of the individual, the cervical bone C7 of the individual, and the cervical bone C3 of the individual when the substance is not blood.

38. The method of claim 25, wherein the body scanning device is an ultrasound device.

39. The method of claim 36, wherein the body scanning device is an ultrasound device.

40. The system of claim 1, further comprising at least one sensor in communication with the CPU, said at least one sensor adapted to detect pressure.

41. The method of claim 37, further comprising the step of at least one of the mechanical arm and the adjustable connecting arm getting pressure feedback from at least one sensor, said sensor transmitting information to the CPU.
